(19) United States
(12) Patent Application Publication
(10) Pub. No.: US 2019/0358417 A1
(43) Pub. Date: Nov. 28, 2019

(54) NASAL DELIVERY DEVICE FOR A POWDER

(71) Applicant: APTAR FRANCE SAS, LE NEUBOURG (FR)

(72) Inventors: Guillaume BROUET, ROUEN (FR); Franck POULLAIN, LA HAYE MALHERBE (FR); Olivier MICHELET, ROUEN (FR)

(73) Assignee: APTAR FRANCE SAS, LE NEUBOURG (FR)

(21) Appl. No.: 16/067,862

(22) PCT Filed: Jan. 6, 2017
(86) PCT No.: PCT/FR2017/050040

§ 371 Date: Sep. 20, 2018

(30) Foreign Application Priority Data
Jan. 7, 2016 (FR) 1650118

(51) Int. Cl. A61M 15/08

(52) U.S. Cl. CPC A61M 15/08; A61M 2202/064

(57) ABSTRACT

A nasal powder dispenser device having a reservoir containing at least one dose of powder; a nasal dispenser head for inserting into a user's nostril, the nasal dispenser head including a dispenser orifice; and an air expeller that, during actuation of the nasal powder dispenser device, generates a flow of compressed air so as to dispense a dose of powder into the nostril through the dispenser orifice. The air expeller has an air chamber and a piston that slides in airtight manner in the air chamber so as to compress the air contained in the air chamber. In the nasal powder dispenser device, a pressure of the flow of compressed air generated by the air expeller is higher than 0.7 bar; and a volume of the air chamber is greater than 1700 mm³.

The present invention relates to a nasal powder dispenser device.

Nasal powder dispenser devices are well known. They generally comprise: a reservoir containing one or more doses of powder; dispenser means; and a nasal dispenser head for inserting into a user's nostril, said nasal dispenser head including a dispenser orifice. The dispenser means generally include an air expeller. When the dispenser device is actuated, a dose of powder is dispensed into a user's nostril.

A drawback with those prior-art devices relates to dispensing the dose of powder into the nostril.

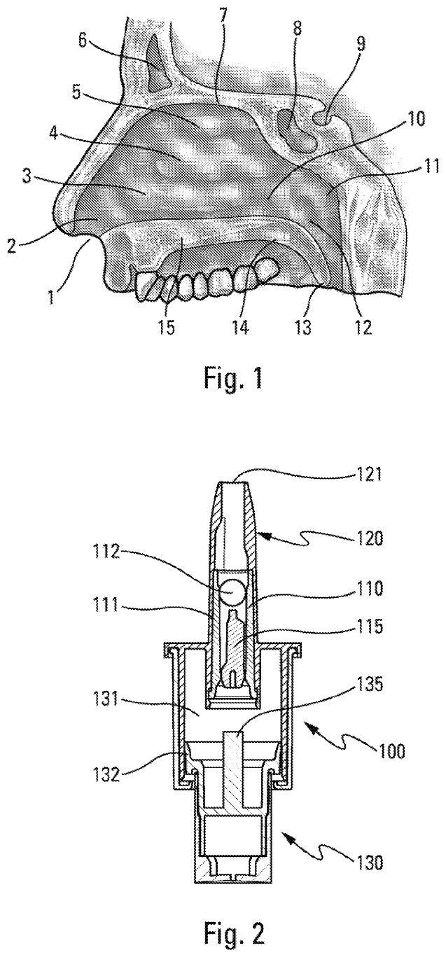

FIG. 1 is a diagram showing such a nostril. The nostril comprises: the nostril orifice 1 (sometimes known as the naris); the nasal valve 2; the inferior concha (or turbinate) 3, the middle concha (or turbinate) 4, and the superior concha (or turbinate) 5; the frontal sinus 6; the ethmoid sinuses 7; the sphenoidal sinus 8, the sella turcica 9, the choana 10, the cavum (or nasopharynx) 11, the Eustachian tube 12, the uvula 13, the soft palate 14, and the hard palate 15.

The nasal valve has a particular shape. It extends over about 1 centimeter (cm) in depth, has a vertical longitudinal section of about 3 cm to 4 cm, and a width of about 1 millimeter (mm) to 3 mm. Beyond the nasal valve, the nasal cavity includes a larger cavity (about 7 cm in height by 2 cm to 3 cm in width). The conchae face the nasal valve. The roof of the nasal cavity is situated above the conchae, which roof includes the ethmoid sinuses, the olfactory bulb, and the olfactory nerve.

Since nasal dispenser devices are non-invasive, or only minimally invasive, the dispensed powder generally does not pass through the nasal valve 2. Thus, as a result of the anatomy of the nasal valve 2 and of the protective location of the conchae 3, 4, 5, the axial or rectilinear path of the powder spray particles does not make it possible to reach the roof of the nasal cavity, and in particular the ethmoid sinuses 7.

Documents WO 99/46055, WO 02/45866, and WO 2015/001281 describe prior-art devices.

An object of the present invention is to provide a nasal powder dispenser device that does not have the above-mentioned drawbacks.

Another object of the present invention is to provide a nasal powder dispenser device that improves the percentage of dose that reaches the ethmoid sinuses.

Another object of the present invention is to provide a nasal powder dispenser device that is simple and inexpensive to manufacture and to assemble.

The present thus provides a nasal powder dispenser device comprising: a reservoir containing at least one dose of powder; a nasal dispenser head for inserting into a user's nostril, said nasal dispenser head including a dispenser orifice; and an air expeller that, during actuation of said nasal powder dispenser device, generates a flow of compressed air so as to dispense a dose of powder into said nostril through said dispenser orifice, said air expeller comprising an air chamber and a piston that slides in airtight manner in said air chamber so as to compress the air contained in said air chamber; said nasal powder dispenser device presenting the following properties:

a pressure, of said flow of compressed air generated by said air expeller, that is higher than 0.7 bar; and

a volume, of said air chamber, that is greater than 1700 cubic millimeters (mm³).

Advantageously, said pressure is lower than 2 bar, in particular lying in the range 1 bar to 1.5 bar, preferably about 1.3 bar.

Advantageously, said volume is smaller than 3000 mm³, in particular lying in the range 2000 mm³ to 2700 mm³, preferably about 2600 mm³.

Advantageously, said dose of powder is 10 milligrams (mg).

Advantageously, the average size of particles of the dose of powder is greater than 5 micrometers (pm).

Advantageously, said reservoir contains a single dose of powder for dispensing during a single actuation of said nasal powder dispenser device.

Advantageously, said nasal powder dispenser device includes a plurality of reservoirs, each containing a single dose of powder.

These characteristics and advantages and others appear more clearly from the following detailed description, given by way of non-limiting examples, and with reference to the accompanying drawings, and in which:

FIG. 1 is a diagrammatic view of a nostril;

FIG. 2 is a diagrammatic view of a nasal powder dispenser device adapted for the present invention; and

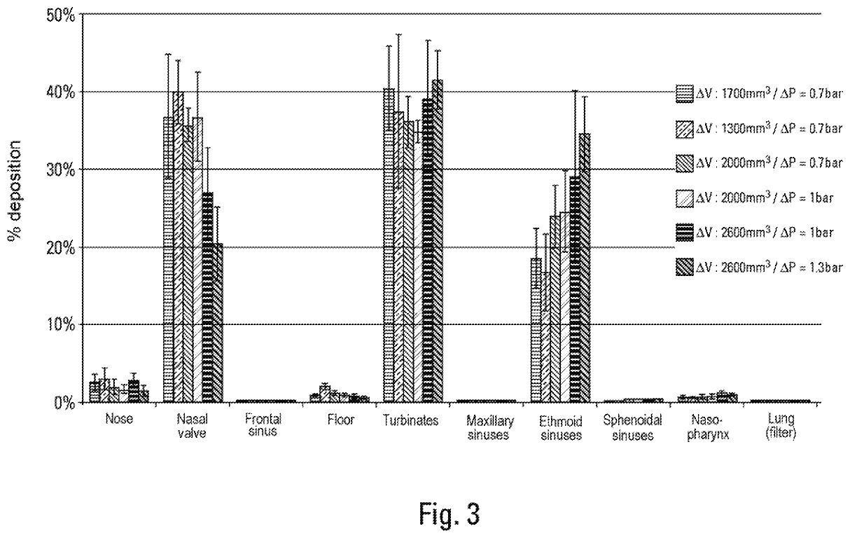

FIG. 3 is a chart showing percentage powder deposition in the various zones of the nostril depending on the volume of the air expeller and on the pressure generated by said air expeller.

In the description, the terms “axial” and “radial” are relative in particular to the longitudinal direction of the device shown in FIG. 2. The terms “proximal” and “distal” are relative to the dispenser orifice of said device.

The invention applies more particularly to devices of the single-powder-dose type, as shown in FIG. 2. Naturally, other types of nasal powder dispenser devices can also be envisaged.

The device 100 shown in FIG. 2 includes a reservoir 110 containing a dose of powder. Devices with a reservoir containing more than one dose are possible. Likewise, devices including a plurality of reservoirs, each containing a single dose are also possible.

A nasal dispenser head 120 is assembled on said reservoir 110, said head being for inserting into a user's nostril. Said nasal dispenser head includes a dispenser orifice 121.

The device 100 further includes an air expeller 130 that, during actuation of said device 100, generates a flow of compressed air so as to dispense a dose of powder into said nostril through said dispenser orifice 121. Said air expeller comprises an air chamber 131 and a piston 132 that slides in airtight manner in said air chamber 131 so as to compress the air contained in said air chamber 131 and thus generate said flow of compressed air.

In the embodiment shown in FIG. 2, the reservoir 110 is formed by a hollow tube 111 that is open at both of its axial ends, and that is closed at its proximal end by a closure element 112, such as a ball, and that is closed at its distal end by an insert 115. The insert 115 includes a rod-forming axial extension, and, during actuation, it can slide in said hollow tube 111 so as to expel said closure element 112 out from its closed position. In this embodiment, the piston 132 of the air expeller 130 is secured to an axial projection 135 that extends in the proximal direction, and that, during actuation, moves together with the piston 132 while the air contained in the air chamber 131 is being compressed. When said projection 135 of the piston 132 comes into contact with said insert 115 of the reservoir 110, a continuation of the movement of the piston 132 causes said insert 115 to slide in said hollow tube 111 out from its closed position. Said insert 115 firstly opens the passage between the air expeller 130 and the reservoir 110, and secondly causes the closure element 112 to be expelled. Thus, the compressed air in the air chamber 131 flows into said reservoir and drives the dose of powder out from said reservoir towards said dispenser orifice 121. Documents WO 99/46055, WO 02/45866, and WO 2015/001281 describe devices of this type. Naturally, other types of dispenser can also be envisaged.

In the invention, the nasal powder dispenser device 100 presents the following properties:

a pressure, of said flow of compressed air generated by said air expeller 130, that is higher than 0.7 bar, advantageously lower than 2 bar, advantageously lying in the range 1 bar to 1.5 bar, preferably about 1.3 bar; and

a volume, of said air chamber 131, that is greater than 1700 mm³, advantageously smaller than 3000 mm³, advantageously lying in the range 2000 mm³ to 2700 mm³, preferably about 2600 mm³.

This combination of pressure and volume of the air expeller makes it possible to optimize the percentage of powder that is deposited on the ethmoid sinuses.

FIG. 3 shows comparative tests demonstrating, for a single dispenser device and for a single dose of powder of 10 mg, that:

with an identical pressure of 0.7 bar, a volume of 2000 mm³ provides better deposition at the ethmoid sinuses compared to volumes of 1300 mm³ and 1700 mm³;

with an identical volume of 2000 mm³, a pressure of 1 bar provides better deposition at the ethmoid sinuses compared to a pressure of 0.7 bar;

with an identical volume of 2600 mm³, a pressure of 1.3 bar provides better deposition at the ethmoid sinuses compared to a pressure of 1 bar; and

the best deposition at the ethmoid sinuses, approximately 35%, is obtained with a volume of 2600 mm³ and a pressure de 1.3 bar.

It should be observed that the pressure and volume values of the air expeller may depend on the dose of powder to be dispensed, and that the maximum values may be limited by the difficulty caused to the user and by the need for actuation of the dispenser device not to be too difficult.

In known manner for nasal powder dispensing, it is desirable for the average size of the particles of the dose of powder to be greater than 5 μm.

The present invention is described with reference to an advantageous implementation, however it should be understood that a person skilled in the art can apply any modification without going beyond the ambit of the present invention as defined by the accompanying claims.

1. A nasal powder dispenser device comprising: a reservoir containing at least one dose of powder; a nasal dispenser head for inserting into a user's nostril, said nasal dispenser head including a dispenser orifice; and an air expeller that, during actuation of said nasal powder dispenser device, generates a flow of compressed air so as to dispense a dose of powder into said nostril through said dispenser orifice, said air expeller comprising an air chamber and a piston that slides in airtight manner in said air chamber so as to compress the air contained in said air chamber; the device being characterized in that said nasal powder dispenser device presents the following properties:
a pressure, of said flow of compressed air generated by said air expeller, that is higher than 0.7 bar; and
a volume, of said air chamber, that is greater than 1700 mm³.

2. A device according to claim 1, wherein said pressure is lower than 2 bar.

3. A device according to claim 1, wherein said pressure lies in the range 1 bar to 1.5 bar.

4. A device according to claim 1, wherein said pressure is about 1.3 bar.

5. A device according to claim 1, wherein said volume is smaller than 3000 mm³.

6. A device according to claim 1, wherein said volume lies in the range 2000 mm³ to 2700 mm³.

7. A device according to claim 1, wherein said volume is about 2600 mm³.

8. A device according to claim 1, wherein said dose of powder is 10 mg.

9. A device according to claim 1, wherein the average size of particles of the dose of powder is greater than 5 μm.

10. A device according to claim 1, wherein said reservoir contains a single dose of powder for dispensing during a single actuation of said nasal powder dispenser device.

11. A device according to claim 10, wherein said nasal powder dispenser device includes a plurality of reservoirs, each containing a single dose of powder.
